(19) United States
(12) Patent Application Publication
(10) Pub. No.: US 2010/0016792 A1
(43) Pub. Date: Jan. 21, 2010

(54) COLLECTING SHEATH AND METHOD OF USE THEREOF

(76) Inventor: Eran Hirszowicz, Ramat-Gan (IL)

Correspondence Address: DANIEL J SWIRSKY, 55 REUVEN ST., BEIT SHEMESH, 99544, IL

(21) Appl. No.: 12/306,934

(22) PCT Filed: Jul. 5, 2007
(86) PCT No.: PCT/IL07/00845

§ 371 Date: Sep. 14, 2009

Related U.S. Application Data
(60) Provisional application No. 60/818,529, filed on Jul. 6, 2006.

(51) Int. Cl. A61M 29/00

(52) U.S. Cl. 604/96.01

(57) ABSTRACT

The present invention is directed to a device and method for safely removing matter collected by a matter collecting catheter from body organ or internal passage by means of a collecting sheath comprising an elongated tube having at a distal end thereof a deformable container sleeve, a main lumen passing therealong, said lumen being suitable for the passage of the matter collecting catheter through said elongated tube and said deformable container sleeve, and a secondary lumen passing along the walls of said elongated tube and said deformable container sleeve, wherein said secondary lumen terminates in a distal opening near a distal opening of said deformable container sleeve such that a distal end portion of a wire passed through said secondary lumen may be adapted to close said distal opening of said deformable container sleeve by proximal retraction of said wire.

### FIELD OF THE INVENTION

The present invention relates to means for removing matter from a body organ or internal passage. More particularly, the invention relates to a device and method for safely removing matter collecting catheter having collected matter gathered from a body organ or internal passage.

### BACKGROUND OF THE INVENTION

Balloon catheters capable of collecting matter (e.g., debris) from a body organ or passage way (e.g., blood vessel) are described in detail in International Patent Applications Serial Nos. WO 2007/004221 and WO 2007/042935 both entitled “balloon catheter”, and in U.S. patent application Ser. No. 11/477,812 entitled “balloon catheter”, filed on Jun. 30, 2006, all of which are of the present assignee, the entire disclosures of which are incorporated herein by reference.

For the sake of clarity the structure and operation of a possible embodiment of balloon catheter 10 (hereinafter also referred to as matter collecting catheter) designed to collect matter 15 from a body organ or internal passage 12 will be briefly described with reference to FIG. 1. Balloon catheter 10 comprises an outer tube 18 containing an inner tube 19 passing thereinside such that a distal end thereof outwardly protrudes via the distal opening of outer tube 18. Balloon 5 may be implemented by an inflatable sleeve having tapering ends configured to tightly fit over tubes 18 and 19 in attachments 7 and 6, respectively. This tube structure defines an annular inflation lumen 16 between tubes 18 and 19 which may be used to inflate balloon 5 with suitable inflation media. As shown in FIG. 1 balloon 5 may be folded inwardly by pulling inner tube 19 proximally and thereafter discharging portions of inflation media from balloon 5, which in turn forms a cavity 14 in the folded balloon structure, that may be advantageously used to collect matter 15 (e.g., debris) from body organ or internal passage 12 (e.g., blood vessel).

However, after collecting matter 15 in balloon 5 the deflated diameter of balloon 5 in its inwardly folded state may be greater than the diameter of the inner lumen of a guiding catheter (30, shown in FIG. 3) through which it should be removed from the body of the treated subject. If balloon 5 is proximally pulled into the guiding catheter it will be squeezed and some of the collected material 15 may be discharged from cavity 14 back into body organ or internal passage 12.

A collection device is described in WO 05/112783, wherein a barrier membrane connected to an expandable clamp is used for collecting a tissue sample. After collecting the tissue sample the clamp is retracted into the collection device such that the barrier membrane connected thereto is changed into a pouch configuration containing the sample tissue.

A modular adjustable blood filter device is described in U.S. Pat. No. 6,152,947. This filter apparatus includes a shaft and a filter frame adjustable between a contracted condition and an enlarged condition, a frame sizing mechanism and a filter mesh coupled thereto. After insertion the frame sizing mechanism adjusts the diameter of the filter frame to conform to the inner lumen of the vessel. The adjustable frame may be closed into a contracted state for entrapping embolic material, and the adjustable filter may be withdrawn into an insertion device.

Another emboli capturing system is described in US 2005/0101986, wherein an expandable emboli capturing device is mounted near the distal end of an elongate member, and is movable between a radially expanded position, in which the emboli capturing device forms a basket with a proximally opening mouth, and a radially contracted position. A delivery device having a receiving end is configured to receive the emboli capturing device and retains at least the mouth of the emboli capturing device in a radially retracted position.

The clot removal device described in WO 02/055146 includes a proximal obstructive matter retaining member having a radially flared and splayable distal end capable of being advanced out of a distal end of a guide catheter such that the clot is captured between the distal obstructive matter receiving portion and the flared distal end of the proximal obstructive matter retaining member.

In US 2002/0121472 an intravascular filter is used for collecting debris dislodged during a medical procedure, wherein an actuatable dilator tip is utilized to retrieve the filter at least in part within a distal sheath. US 2002/0082639 describes an emboli capturing system which includes an expandable member having spaced portion spaced radially outwardly from a guide-wire, and emboli capturing device being coupled to an expandable member.

There is still a need for suitable means for safely removing matter collecting catheter having collected matter gathered from body organ or internal passage.

It is therefore an object of the present invention to provide a device and method for removing matter collected from a body organ or internal passage.

It is another object of the present invention to provide a device and method for holding, and preventing the discharge of, matter collected by a matter collecting catheter form a body organ or internal passage.

Other objects and advantages of the invention will become apparent as the description proceeds.

### SUMMARY OF THE INVENTION

The present invention is directed to a device and method for safely removing matter collected by a matter collecting catheter from body organ or internal passage. The inventor of the present invention found out that matter collected by means of a matter collecting catheter from a body organ or passage can be safely removed from the body of the treated subject by means of a collecting sheath comprising an elongated tube having at its distal end a deformable container sleeve, a main lumen passing therethrough, said lumen being suitable for the passage of the matter collecting catheter through said elongated tube and said deformable container sleeve, and a secondary lumen passing along the walls of said elongated tube and said deformable container sleeve, wherein said secondary lumen terminates in a distal opening near a distal opening of said deformable container sleeve such that a distal end portion of a wire passed through said secondary lumen may be adapted to close said distal end opening of said deformable container sleeve by proximal retraction thereof.

The deformable container sleeve may be introduced into the body organ or passage in a squeezed state for reducing its cross-sectional area. The matter collecting catheter may be introduced before or after introducing the collecting sheath into the body organ or passage, or alternatively it may be contained in its first lumen during insertion thereof.

The collecting sheath may be introduced into the body organ or passage over a guidewire, over a previously introduced matter collecting catheter, or via a guiding catheter.

After collecting material from the body organ or passage by the material collecting catheter, it is introduced into the main lumen of the deformable container sleeve through a distal opening, thereby forcing it to radially expand and open into an un-squeezed state. Thereafter, the collecting sheath and the material collecting catheter contained in its main lumen are retracted proximally and removed from the body of the treated subject.

In a preferred embodiment of the invention, the device is used to collect atherosclerotic debris from an intravascular lesion, such as may commonly be found in the coronary arteries or in saphenous vein grafts. It is to be recognized, however, that the use of the presently-disclosed device is not limited to any particular vessel or passage, but rather may be applied to any body organ or internal passage.

In one aspect the present invention is directed to a device for removing matter collected by a matter collecting catheter from a body organ or passage, said device comprises an elongated tube having at its distal end a deformable container sleeve, and main and secondary lumens passing therethrough, wherein said first lumen is adapted to permit passage of said matter collecting catheter through said elongated tube and said deformable container sleeve, and wherein said secondary lumen passes along the walls of said elongated tube and said deformable container sleeve and terminates in a distal opening near a distal opening of said deformable container sleeve, such that a distal end portion of a wire passed through said secondary lumen may be adapted to close said distal opening of said deformable container sleeve by proximal retraction hereof.

Preferably, the deformable container sleeve is introduced into the body organ or passage in a squeezed state. The deformable container sleeve may be expanded radially into an un-squeezed state by introducing into the main lumen via the distal opening of the deformable container sleeve a distal end portion of the matter collecting catheter having matter collected thereby.

The secondary lumen may be formed in the walls of the elongated tube and of the deformable container sleeve. Preferably, a distal section of the secondary lumen passes through a thin tube passing long the length of the deformable container sleeve.

The secondary lumen is adapted to permit passage of a wire therethrough, such that a distal end portion of said wire may leave said secondary lumen via a distal opening thereof, said distal end portion of said wire may be wrapped around, or threaded through, an annular distal end section of said deformable container sleeve, thereby forming a strangling loop (e.g., in a form of a lasso) which may be used to close the distal end opening of said deformable container sleeve by pulling said wire proximally.

Preferably, the container sleeve is made from a porous material, such as a mesh, comprising pores which are smaller that the particle size of the matter to be removed therewith.

In a specific embodiment of the invention a balloon catheter device is employed for collecting matter from the body organ or passage by means of an invaginated balloon. Advantageously, the length of deformable container sleeve is slightly greater than half of the length of the balloon in its invaginated state.

In another aspect the present invention is directed to a method for removing material collected by a matter collecting catheter from a body organ or passage, the method comprising: introducing into said body organ or passage a device comprising an elongated tube having at its distal end a deformable container sleeve being in a squeezed state, and main and secondary lumens passing therethrough, wherein said main lumen is adapted to permit the passage of said matter collecting catheter through said elongated tube and said deformable container sleeve, and wherein said secondary lumen passes along the walls of said elongate tube and said deformable container sleeve and terminates in a distal opening near a distal opening of said deformable container sleeve, such that a distal end portion of a wire passing through said secondary lumen is adapted to close said distal end opening of said deformable container sleeve by proximal retraction thereof; introducing into said main lumen via said distal opening of said deformable container sleeve a distal end portion of said matter collecting catheter having matter collected thereby, thereby radially expanding it into an un-squeezed state; closing said distal end opening of said deformable container sleeve by retracting said wire proximally; and removing said device and the matter collecting catheter contained in its main lumen from the body of the treated subject.

### BRIEF DESCRIPTION OF THE DRAWINGS

The present invention is illustrated by way of example in the accompanying drawings, in which similar references consistently indicate similar elements and in which:

FIG. 1 schematically illustrates a possible implementation of a balloon catheter suitable for collecting matter from a body organ or internal passage;

FIG. 2A schematically illustrates a preferred embodiment of a collecting sheath device of the invention;

FIG. 2B is cross-sectional view of the tube of the collecting sheath device of the invention;

FIG. 3 schematically illustrates introducing the balloon catheter and the collecting sheath device via a guiding catheter;

FIG. 4 schematically illustrates the balloon catheter and collecting sheath device after collecting matter by the balloon;

FIG. 5 schematically illustrates inserting the balloon catheter into a container sleeve of the collecting sheath after collecting matter therewith; and

FIG. 6 schematically illustrates the container sleeve of the collecting sheath of the invention, comprising the folded balloon, after its distal end opening is closed.

### DETAILED DESCRIPTION OF PREFERRED EMBODIMENTS

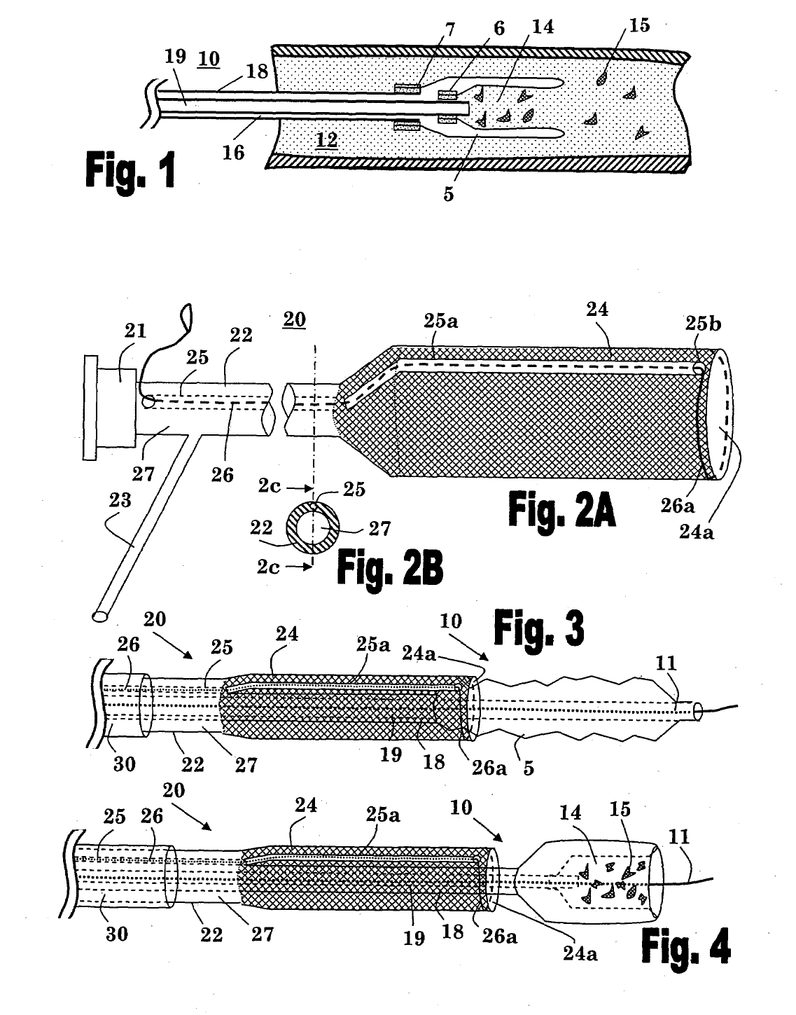

The present invention is directed to a device and method for safely removing matter collected from a body organ or internal passage. While the collected matter may be obtained in various ways, in one preferred embodiment of the invention it is sucked into a cavity formed in an invaginated balloon of a balloon catheter device by folding a distal portion thereof inwardly as exemplified hereinabove with reference to FIG. 1. The device of the invention is designed to withstand forces during retraction which squeeze the inwardly folded balloon containing the collected matter via a relatively narrow guiding catheter without discharging collected matter back into the treatment site or elsewhere in the body.

In general, the matter removing device (also referred to herein as collecting sheath) of the invention comprises an elongated tube comprising two lumens, and a container sleeve connected to the distal end opening of the main lumen of said elongated tube. The main lumen of the collecting sheath is designed to permit passage of the balloon catheter therethrough.

The secondary lumen passing in collecting sheath, which is of a substantially small diameter, is designed to permit passage of a tying wire therethrough. A small diameter tube passing along the length of the container sleeve is connected to the distal end opening of the secondary lumen passing in the collecting sheath. A tying wire is threaded through the passage provided by the secondary lumen and the small diameter tube connected to it, and a distal end portion thereof leaves said passage at the distal end opening of the small diameter tube, near the distal end opening of the container sleeve. The distal end portion of the tying wire is wrapped a round, or threaded through, the distal end portion of the container sleeve forming a “strangling” loop therearound (e.g., in a form of a lasso) such that it may be used to close its distal end opening by pulling said tying wire proximally.

The distal end of the wire may be connected to the edge of the distal end of the container sleeve, or alternatively the wire may be threaded back proximally, such that both of its extremities are used to tighten a strangling loop formed by an intermediate portion thereof, which is wrapped around, or threaded through, the distal end portion of the container sleeve.

The container sleeve is advantageously made from mesh having pores which should be slightly smaller than the size of the particulate matter to be collected therewith. In this way matter collected from a body organ or internal passage may be sealed in the container sleeve by pulling said tying wire and closing the distal end opening of the container sleeve, while permitting fluids to leave the interior of container sleeve via the mesh pores in its wall.

The collecting sheath containing the balloon catheter is preferably introduced into the body of the treated subject via a guiding catheter in a state in which its container sleeve is in a squeezed state to assume a reduced cross-sectional profile. After reaching the treatment site the distal part of balloon catheter containing the balloon is advanced outwardly via the distal opening of container sleeve for performing the requisite treatment and collecting matter obtained therein. The container sleeve may be then opened into its un-squeezed state by retracting the distal part of balloon catheter proximally thereinto, such that the inwardly folded balloon is pressed thereinto and thus radially push the wall of squeezed container sleeve into an open (un-squeezed) state.

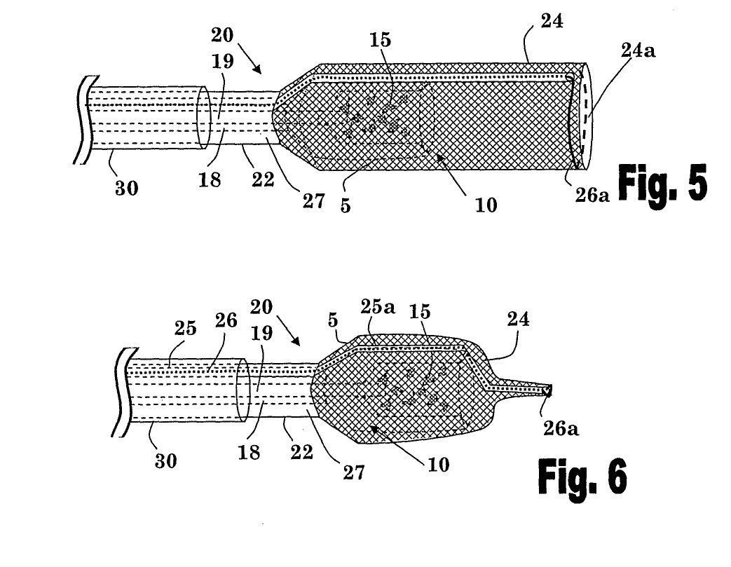

FIG. 2A schematically illustrates a preferred embodiment of a collecting sheath 20 of the invention which may be used to remove matter collected from a body organ or internal passage. Collecting sheath 20 comprises an elongated tube 22 comprising two lumens, 27 and 25, passing along its length. The main lumen 27 of tube 22 is of a diameter that permits threading it over balloon catheter 10 (shown if FIGS. 3 to 6). The distal end opening of main lumen 27 is connected to container sleeve 24, which is configured to receive the balloon catheter 10, after the collecting matter therewith from the treatment site, and safely remove it therefrom through the guiding catheter (30) out of the body of the treated subject. The length of container sleeve 24 should be longer than the length of balloon 5 when in its folded state (shown in FIG. 1).

The proximal end opening of main lumen 27 is sealed by a proximal seal 21 which is used to prevent outflow of fluids (e.g., blood) therefrom during the treatment procedure, or of flushing fluids obtained therein when the device is flushed via optional flushing tube 23. Optional flushing tube 23 is connected to tube 22 and allows repeated flushing of the sheath by suitable fluids (e.g., heparinized saline), before or after treatment.

Secondary lumen 25 may be formed in the wall of tube 22 as exemplified in the cross-sectional view shown in FIG. 2B, and it may be accessed via a lateral proximal end opening provide on tube 22. The distal end of secondary lumen 25 is connected to a small diameter tube 25a attached to, or passing in, container tube 24. Tying wire 26 is threaded through the passage obtained between secondary lumen 25 and small diameter tube 25a, and a distal end portion thereof 26a leaves small diameter tube 25a via its distal end opening and is threaded into (or wrapped around) an annular distal end section of container sleeve 24 forming a strangling loop (e.g., in the form of a lasso) therearound.

FIG. 3 schematically illustrates introducing the balloon catheter 10 and collecting sheath 20 into the treatment site (not shown) via a guiding catheter 30. The collecting sheath 20 may be advanced through guiding catheter 30 towards the treatment site while balloon catheter 10 is contained in its main lumen 27. Alternatively, collecting sheath 20 may be kept proximally ready for insertion into the treatment site and allowing insertion of balloon catheter 10 therethrough, such that it may be threaded over balloon catheter 10 in a later stage of the procedure. As exemplified in FIG. 3, during insertion into the body of the treated subject container sleeve 24 is in a squeezed state, to permit easy advancement through guiding catheter 30; balloon 5 is similarly in a deflated state.

Generally, debris or other particulate matter is collected by balloon 5 by inflating it at the treatment site, pulling inner tube 19 proximally, to inwardly fold balloon 5, and thereafter discharging portions of the inflation media from balloon 5 to form cavity 14, into which matter 15 is gathered due to the suction effect obtained. Guidewire 11, threaded through the lumen of inner tube 19, may be used to maneuver balloon catheter 10 in the body of treated subject. FIG. 4 schematically illustrates balloon catheter 10 and collecting sheath 20 after collection of matter 15 from the treatment site by balloon 5.

After collection of matter 15 by balloon 5, the operator may move balloon 5 and collection sheath 20 progressively closer to each other until balloon 5 enters into the internal cavity of container sleeve 24 which is thereby caused to open into its un-squeezed state, as shown in FIG. 5. The operator can then pull tying wire 26 proximally and thereby tighten the strangling loop formed by its distal end portion 26a over the distal end section of container sleeve 24 and thereby close its distal end opening 24a.

Thereafter, collecting sheath 20 and balloon catheter 10 are pulled backward (proximally) (not shown) into guiding catheter 30. In effect, container sleeve 24 and balloon 5 are squeezed into guiding catheter 30, during which a portion of the collected matter 15 may escape from cavity 14 into container sleeve 24. However, this portion of discharged matter remains in container sleeve 24 since its distal end opening 24a is sealably closed by tying wire 26. The collection sheath 20, comprising balloon catheter 10 and collected matter 15 inside it, is then pulled backward through guiding catheter 30 until it is safely removed from the body of the treated subject.

Elongated tube 22 of collection sheath 20 may be manufactured from any suitable biocompatible material such as nylon 12 or pebax or from a similar compound. Container sleeve 24 can be made from silk mesh, polyurethane mesh, or metallic mesh, having pore size in the range of 10 to 200 microns, preferably in the range of 50 to 80 microns, and wall thickness of about 5-20 microns. The mesh wall of container sleeve 24 may be reinforced by the addition of glass fibers, carbon fibers materials, or other additives suitable for preventing buckling thereof. The surfaces of collection sheath 20 may be treated (or suitable additives may be added to the materials it is made from) in order to reduce its surface friction. Some radio opaque markers may be added to the distal portion of collection sheath 20 to enhance imaging during the procedure.

The inner diameter of tube 22 should be compatible with the outer diameter of balloon 5 of balloon catheter 10 when it is in its deflated state, which is about 0.5 mm to 1.5 mm. The wall thickness of collection sheath 20 may vary between 0.05 mm to 0.2 mm. The length of collection sheath 20 is preferably slightly shorter than the length of balloon catheter 10, which is about 0.7 meter to 1.8 meter.

The length of container sleeve 24 is generally between 2 mm to 10 mm longer than half of the length of balloon 5 in its unfolded state. The inner diameter of container sleeve 24 is preferably the same as, or slightly greater than, the outer diameter of the balloon 5 in its inflated state. The wall thickness of container sleeve 24 should be as small as possible in order to reduce its cross-section profile and still retain stability, for example, it may be in the range of 5 microns to 50 microns.

Tying wire may be any type of wire made from metal, such as stainless steel. The diameter of tying wire may generally be in the range of 2 to 20 microns, preferably about 5 microns. The inner diameter of tube 22 is generally in the range of 1 to 3 mm, preferably about 1.5 mm, and its wall thickness is generally in the range of 0.1 to 0.3 mm, preferably about 0.2 mm. The inner diameter of secondary lumen 25 is generally in the range of 0.01 to 0.2 mm, preferably about 0.1 mm. Small diameter tube 25a may be a polymer tube made from a type of polyurethane, such as pelethan. The inner diameter of small diameter tube 25a may generally be in the range of 0.01 to 0.3 mm, preferably about 0.05 mm, and its wall thickness may generally be in the range of 0.01 to 0.05 mm, preferably about 0.05 mm.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different tubes, balloons, sleeves, and other members, described hereinabove may be constructed in different shapes (e.g. having oval, square etc. form in plan view) and sizes differing from those exemplified in the preceding description.

The above description have of course has been provided only for the purpose of illustration, and is not intended to limit the invention in any way. As will be appreciated by the skilled person, the invention can be carried out in a great variety of ways, employing more than one technique from those described above, all without exceeding the scope of the invention.

1. A device for removing matter collected by a matter collecting catheter from a body organ or passage, comprising an elongated tube having at its distal end a deformable container sleeve, and main and secondary lumens passing therethrough, wherein said first lumen is adapted to permit passage of said matter collecting catheter through said elongated tube and said deformable container sleeve, and wherein said secondary lumen passes along the walls of said elongated tube and said deformable container sleeve and terminates in a distal opening near a distal opening of said deformable container sleeve, such that a distal end portion of a tying wire passed through said secondary lumen may be adapted to close said distal end opening of said deformable container sleeve by proximal retraction thereof.

2. The device according to claim 1, wherein the deformable container sleeve is introduced into the body organ or passage in a squeezed state.

3. The device according to claim 1, wherein the deformable container sleeve may be expanded radially into an un-squeezed state by introducing into the main lumen via the distal opening of the deformable container sleeve a distal end portion of the matter collecting catheter.

4. The device according to claim 1, wherein the secondary lumen may be formed in the walls of the elongated tube and of the deformable container sleeve.

5. The device according to claim 1, wherein a distal section of the secondary lumen passes through a thin tube passing long the length of the deformable container sleeve.

6. The device according to claim 1, wherein the secondary lumen is adapted to permit passage of a wire therethrough, such that a distal end portion of said wire is capable of leaving said secondary lumen via a distal opening thereof, and capable of being wrapped around, or threaded through, an annular distal end section of said deformable container sleeve.

7. The device according to claim 1, wherein the deformable container sleeve is made from a porous material.

8. The device according to claim 7, wherein the deformable container sleeve is made from a material comprising pores which are smaller that the particle size of the matter to be removed therewith.

9. The device according to claim 1, wherein the matter collecting catheter is a balloon catheter device.

10. The device according to claim 9, wherein the balloon catheter device is capable of collecting matter from the body organ or passage by means of an invaginated balloon.

11. The device according to claim 10, wherein the length of the deformable container sleeve is slightly greater than half of the length of the balloon in its invaginated state.

12. The device according to claim 1, wherein a guiding catheter is used for introducing/removing said device into/from the body of the treated subject.

13. A method for removing material collected by a matter collecting catheter from a body organ or passage, the method comprising: introducing into said body organ or passage a device comprising an elongated tube having at its distal end a deformable container sleeve being in a squeezed state, and main and secondary lumens passing therethrough, wherein said main lumen is adapted to permit passage of said matter collecting catheter through said elongated tube and said deformable container sleeve, and wherein said secondary lumen passes along the walls of said elongate tube and said deformable container sleeve and terminates in a distal opening near a distal opening of said deformable container sleeve, such that a distal end portion of a wire passing through said secondary lumen is adapted to close said distal opening of said deformable container sleeve by proximal retraction of said wire; introducing into said main lumen via said distal opening of said deformable container sleeve said matter collecting catheter having matter collected thereby, thereby radially expanding it into an un-squeezed state; closing said distal opening of said deformable container sleeve by retracting said wire proximally; and removing said device and the matter collecting catheter contained in its main lumen from the body of the treated subject.

14. The method according to claim 13, wherein the closing of the distal opening of the deformable container sleeve is affected by a strangling loop formed by a distal portion of the wire around, or through, an annular distal end section of said deformable container sleeve.
